SEQUENCE LISTING (16 sequences)

SEQ ID NO: 1 - protein, 8 aa, Artificial Sequence
  Description of Artificial Sequence Synthetic peptide
  Linker sequence
GGGSGGGG

SEQ ID NO: 2 - protein, 5 aa, Artificial Sequence
  Description of Artificial Sequence Synthetic peptide
  Linker sequence
GGGGS

SEQ ID NO: 3 - protein, 5 aa, Artificial Sequence
  Description of Artificial Sequence Synthetic peptide
  Linker sequence
GGGSG

SEQ ID NO: 4 - protein, 5 aa, Artificial Sequence
  Description of Artificial Sequence Synthetic peptide
  Linker sequence
GGSGG

SEQ ID NO: 5 - protein, 5 aa, Artificial Sequence
  Description of Artificial Sequence Synthetic peptide
  Linker sequence
GSGGG

SEQ ID NO: 6 - protein, 6 aa, Artificial Sequence
  Description of Artificial Sequence Synthetic peptide
  Linker sequence
GSGGGP

SEQ ID NO: 7 - protein, 5 aa, Artificial Sequence
  Description of Artificial Sequence Synthetic peptide
  Linker sequence
GGEPS

SEQ ID NO: 8 - protein, 7 aa, Artificial Sequence
  Description of Artificial Sequence Synthetic peptide
  Linker sequence
GGEGGGP

SEQ ID NO: 9 - protein, 12 aa, Artificial Sequence
  Description of Artificial Sequence Synthetic peptide
  Linker sequence
GGEGGGSEGGGS

SEQ ID NO: 10 - protein, 226 aa, Homo sapiens
YGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLYCLVKGFYPSDIAVEWESNGQPENNYKTTPPVLDSDGSFFLYSRLTVDKSRWQEGNVFSCSVMHEALHNHYTQKSLSLSPGK

SEQ ID NO: 11 - protein, 226 aa, Homo sapiens
YGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLTSRLTVDKSRWQEGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 223 aa, Homo sapiens
VECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 14 - protein, 20 aa, Homo sapiens
QRLMEDICLPRWGCLWEDDF

SEQ ID NO: 15 - protein, 450 aa, Homo sapiens
GDAKTTQPNSMESNEEEPVHLPCNHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSTGGGGSGGGGSGGGRSGTVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 743 aa, Homo sapiens
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGSAPIRFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADTGGGGSGGGGSGGGRSGTVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

(19) United States
(12) Patent Application Publication
(10) Pub. No.: US 2022/0119479 A1
(43) Pub. Date: Apr. 21, 2022

(54) HALF-LIFE EXTENDED IMMTAC BINDING CD3 AND A HLA-A*02 RESTRICTED PEPTIDE

(71) Applicant: IMMUNOCORE LIMITED, Abingdon, Oxfordshire (GB)

(72) Inventors: Paul CONROY, Abingdon, Oxfordshire (GB); Stephen HEARTY, Abingdon, Oxfordshire (GB); Amandine GEORGES, Abingdon, Oxfordshire (GB); Lok Hang MAK, Abingdon, Oxfordshire (GB); Nikolai LISSIN, Abingdon, Oxfordshire (GB); Andrew David JOHNSON, Abingdon, Oxfordshire (GB); Emma HODGSON, Abingdon, Oxfordshire (GB); Udofoyo UYE, Abingdon, Oxfordshire (GB)

(21) Appl. No.: 17/427,581

(22) PCT Filed: Jan. 30, 2020
(86) PCT No.: PCT/EP2020/052316

§ 371 Date: Jul. 30, 2021

(30) Foreign Application Priority Data
Jan. 30, 2019 (GB) 1901306.9

(51) Int. Cl. C07K 14/725; C07K 16/28

(52) U.S. Cl. CPC C07K 14/7051; C07K 16/2809; A61K 2039/505; C07K 2317/94; C07K 2319/30; C07K 2317/622

(57) ABSTRACT

The present invention relates to soluble multi-domain binding molecules comprising T cell receptors (TCR) having specificity for an antigen, an immunoglobulin Fc domain or an albumin-binding moiety; and an immune effector domain. Such multi-domain binding molecules are advantageous because they display improved half-life while retaining function.

The present invention relates to soluble multi-domain binding molecules comprising T cell receptors (TCR) having specificity for an antigen, an immunoglobulin Fc domain or an albumin-binding moiety; and an immune effector domain. Such multi-domain binding molecules are advantageous because they display improved half-life while retaining function.

### BACKGROUND TO THE INVENTION

Many protein-based therapeutics, including antibodies fragments and fusions proteins, are rapidly cleared from the body following administration. Their short circulatory half-life is typically attributed to their small size, which allows for effective clearance via renal filtration, and lack of protection from intracellular degradation. In such cases, frequent administration or long infusion times are required to maintain an effective concentration of the drug over prolonged periods. To improve dosing, several strategies have been employed to extend circulatory half-life. These include increasing the hydrodynamic radius of the protein though attachment of flexible hydrophilic molecules, such as carbohydrate or PEG (polyethelene glycol), and exploiting recycling via the neonatal Fc receptor (FcRn), through attachment of antibody Fc domains or serum albumin (Konnteman, Curr Opin Biotechnol. 2011 December; 22(6):868-76).

Strategies that exploit FcRn mediated recycling are particularly attractive because of the lower risk of inducing immunogenicity in vivo and long half-life extensions that may be achieved. For example, the half-life of a T cell engaging bispecific antibody of the BiTE® format, is reported to be in excess of 200 h, following attachment of an Fc domain (Lorenczewski, et al., Blood 2017. 130 (Suppl 1), 2815). Similarly, bispecific antibodies of the TriTac® format, which incorporate an albumin binding domain, are reported to have a half-life of over four days (Wesche et al., Cancer Res 2018; 78 (13 Suppl): Abstract nr 3814).

Fusions proteins comprising a soluble T cell receptor fused to an anti-CD3 antibody fragment are a novel category of T cell engaging bispecific fusion proteins with an in vivo half-life in the region of 6-8 h (Sato et al., 2018 J Clin Onc 2018 36, no. 15_suppl 9521-9521; Middleton et al., J Clin Onc 2016 34, no. 15_suppl 3016-3016). Unlike traditional antibodies, T cell receptors are designed to recognise short peptides derived from intracellular antigens and presented on the cell surface by human leukocyte antigen (peptide-HLA). Effective immune synapse formation between a peptide-HLA complex on an antigen presenting cell and a T cell relies on a fixed interaction geometry, which is perturbed by increases in intramembrane distance (Choudhuri et al., 2005 Nature July 28; 436(7050):578-82).

### DESCRIPTION OF THE INVENTION

There is a need for T cell engaging bispecific proteins with increased half-life that are able to mediate effective immune synapse formation. Contrary to the expectations in the art, the inventors have found that fusing a TCR-anti-CD3 fusion protein to an antibody Fc region or an albumin binding moiety surprisingly resulted in effective immune synapse formation.

In a first aspect, there is provided a multi-domain binding molecule comprising:
i) a peptide-major histocompatibility complex (pMHC) binding moiety linked to a T cell engaging immune effector; and
ii) a half-life extending domain, comprising an immunoglobulin Fc or an albumin binding domain.

Preferably, the pMHC binding moiety is a T cell receptor (TCR) or TCR-like antibody, comprising TCR and/or antibody variable domains, and at least one constant domain. Preferably the pMHC binding moiety comprises at least one immunoglobulin constant domain. Preferably the constant domain may correspond to a constant domain from a TCR alpha chain or a TCR beta chain (TRAC or TRBC respectively). Alternatively the TCR constant domain of the pMHC binding moiety may be replaced with a constant domain from an antibody light or heavy chain (CL, CH1, CH2, CH3 or CH4). The constant domain may be full length or may be truncated. The TCR constant domain may be truncated to remove the transmembrane domain. Where the constant domain is truncated, preferably only membrane-associated portions are removed. Additional mutations may be introduced in to the amino acid sequence of the constant domain relative to a natural constant domain. The constant region may also include residues, either naturally-occurring or introduced, that allow for dimerization by, for example, a disulphide bond between two cysteine residues.

The present inventors have unexpectedly found that a multi-domain binding molecule comprising a pMHC binding moiety, an immune effector domain and either an immunoglobin Fc domain or an albumin-binding moiety remains functional. This is particularly surprising given the knowledge in the art that the TCR-pMHC interaction relies on a fixed binding geometry and that TCR triggering is sensitive to increases in intermembrane distance (Garboczi et al., Nature. 1996 Nov. 14; 384(6605):134-41; Choudhuri et al., 2005 Nature Jul. 28; 436(7050):578-82; Rudolph et al., Annu Rev Immunol. 2006; 24:419-66). Indeed, the kinetic segregation model proposes that TCR triggering is the result of tethering of the TCR-CD3 complex within close-contact zones in which tyrosine phosphorylation is favoured because of size-dependent exclusion of tyrosine phosphatases such as CD45 (Choudhuri et al., 2005 Nature July 28; 436(7050):578-82; Davis et al., Nat Immunol. 2006 August; 7(8):803-9). The small dimensions of the TCR-pMHC complex (approx. 100 Å) and fixed binding geometry are therefore understood to be important for immune synapse formation and TCR triggering. Based on this knowledge a skilled person would understand that the antigen binding polypeptides of the invention would be expected to result in poor immune synapse formation, perturbation of the TCR-pMHC binding geometry, and ultimately ineffective TCR triggering.

The pMHC binding moiety may be a TCR-like antibody. Preferably the pMHC binding moiety comprises the variable domains of a TCR-like antibody. Antibodies do not naturally recognise a pMHC; however, it is known that antibodies with specificity for pMHC can be engineered. Such antibodies are referred to as TCR-like or TCR-mimic antibodies (Chang et al., Expert Opin Biol Ther. 2016 August; 16(8):979-87 and Dahan et al., Expert Rev Mol Med. 2012 Feb. 24; 14:e6).

The TCR may be a heterodimeric alpha/beta or gamma/delta TCR polypeptide pair. Alternatively, the TCR may be a single chain alpha/beta or gamma/delta TCR polypeptide. The amino acid sequence of the TCR variable domains may correspond to those found in nature, or they may contain one or more mutations relative to a natural TCR. Such mutations may be made to increase the affinity of the TCR for a given antigen. Additionally or alternatively mutations may be incorporated improve stability and manufacturability.

The TCR may bind to MHC in complex with a peptide antigen. Preferably, the peptide antigen is any disease associated antigen. Preferably, the peptide antigen is any tumour associated antigen. The peptide antigen may be a peptide derived from GP100, NYESO, MAGEA4, or PRAME as described in WO2011001152, WO2017109496, WO2017175006 and WO2018234319.

The TCR may have an amino acid sequence as defined in WO2011001152, WO2017109496, WO2017175006 and WO2018234319.

The T-cell engaging immune effector domain may be a CD3 effector domain. The T cell engaging immune effector may be an antibody scFv (or a similar sized antibody-like scaffold) that activates a T cell through interaction with CD3 and or TCR/CD3 complex. CD3 effectors include but are not limited to anti-CD3 antibodies or antibody fragments, in particular an anti-CD3 scFv or antibody-like scaffolds. Further immune effectors include but are not limited to, cytokines, such as IL-2 and IFN-γ; superantigens and mutants thereof; chemokines such as IL-8, platelet factor 4, melanoma growth stimulatory protein; antibodies, including fragments, derivatives and variants thereof, that bind to antigens on immune cells such as T cells or NK cell (e.g. anti-CD28 or anti-CD16 or any molecules that locate to the immune synapse), and complement activators.

The half-life extending domain may be linked to the C or N terminus of the pMHC binding moiety or to the C or N terminus of the T cell engaging immune effector.

The half-life extending domain may comprise an immunoglobulin Fc. The immunoglobulin Fc domain may be any antibody Fc region. The Fc region is the tail region of an antibody that interacts with cell surface Fc receptors and some proteins of the complement system. The Fc region typically comprises two polypeptide chains both having two or three heavy chain constant domains (termed CH2, CH3 and CH4), and a hinge region. The two chains being linked by disulphide bonds within the hinge region. Fc domains from immunoglobulin subclasses IgG1, IgG2 and IgG4 bind to and undergo FcRn mediated recycling, affording a long circulatory half-life (3-4 weeks). The interaction of IgG with FcRn has been localized in the Fc region covering parts of the CH2 and CH3 domain. Preferred immunoglobulin Fc domain for use in the present invention include, but are not limited to Fc domains from IgG1 or IgG4. Preferably the Fc domain is derived from human sequences. The Fc region may also preferably include KiH mutations which facilitate dimerization, as well as mutations to prevent interaction with activating receptors i.e. functionally silent molecules. The immunoglobulin Fc domain may be fused to the C or N terminus of the other domains (i.e., the TCR or immune effector). The immunoglobulin Fc may be fused to the other domains (i.e., the TCR or immune effector) via a linker, alternatively no linker may be used. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules of the invention include, but are not limited to: GGGSGGGG, GGGGS, GGGSG, GGSGG, GSGGG, GSGGGP, GGEPS, GGEGGGP, and GGEGGGSEGGGS (as described in WO2010/133828). Where the immunoglobulin Fc is fused to the TCR, it may be fused to either the alpha or beta chains or both the alpha and beta chains, with or without a linker. Furthermore, individual chains of the Fc may be fused to individual chains of the TCR.

Preferably the Fc region may be derived from the IgG1 or IgG4 subclass. The two chains may comprise CH2 and CH3 constant domains and all or part of a hinge region. The hinge region may correspond substantially or partially to a hinge region from IgG1, IgG2, IgG3 or IgG4. The hinge may comprise all or part of a core hinge domain and all or part of a lower hinge region. Preferably, the hinge region contains at least one disulphide bond linking the two chains.

The Fc region may comprise mutations relative to a WT Fc sequence. Mutations include substitutions, insertions and deletions. Such mutations may be made for the purpose of introducing desirable therapeutic properties. For example, to facilitate heterodimersation, knobs into holes (KiH) mutations maybe engineered into the CH3 domain. In this case, one chain is engineered to contain a bulky protruding residue (i.e. the knob), such as Y, and the other is chain engineered to contain a complementary pocket (i.e. the hole). Suitable positions for KiH mutations are known in the art. Additionally or alternatively mutations may be introduced that abrogate or reduce binding to Fcy receptors and or increase binding to FcRn, and/or prevent Fab arm exchange, or remove protease sites. Additionally or alternatively mutations may be made for manufacturing reasons, for example to remove or replace amino acids that may be subject to post translations modifications such as glycosylation.

Examples include:

IgG4 (underlined residues indicate mutations versus the wildtype sequence)

“Knob”
YGPPCPPCPAPEFEGGPSVFLFPPKPKDTLMISR
TPEVTCVVVDVSQEDPEVQFNVVYVDGVEVHNAK
TKPREEQFNSTYRVVSVLTVLHQDWLNGKEYKCK
VSNKGLPSSIEKTISKAKGQPREPQVYTLPPSQE
EMTKNQVSLYCLVKGFYPSDIAVEWESNGQPENN
YKTTPPVLDSDGSFFLYSRLTVDKSRWQEGNVFS
CSVMHEALHNHYTQKSLSLSPGK
“Hole”
YGPPCPPCPAPEFEGGPSVFLFPPKPKDTLMISR
TPEVTCVVVDVSQEDPEVQFNVVYVDGVEVHNAK
TKPREEQFNSTYRVVSVLTVLHQDWLNGKEYKCK
VSNKGLPSSIEKTISKAKGQPREPQVYTLPPSQE
EMTKNQVSLTCLVKGFYPSDIAVEWESNGQPENN
YKTTPPVLDSDGSFFLTSRLTVDKSRWQEGNVFS
CSVMHEALHNHYTQKSLSLSPGK

IgG1 (underlined residues indicate mutations versus the wildtype sequence)

“Knob”
VECPPCPAPPVAGPSVFLFPPKPKDTLMISRTPEVT
CVVVDVSHEDPEVKFNVVYVDGVEVHNAKTKPREEQ
YNSTYRVVSVLTVLHQDWLNGKEYKCKVSNKGLPSS
IEKTISKAKGQPREPQVYTLPPSREEMTKNQVSLYC
LVKGFYPSDIAVEWESNGQPENNYKTTPPVLDSDGS
FFLYSKLTVDKSRWQQGNVFSCSVMHEALHNHYTQK
SLSLSPGK
“Hole”
VECPPCPAPPVAGPSVFLFPPKPKDTLMISRTPEVT
CVVVDVSHEDPEVKFNVVYVDGVEVHNAKTKPREEQ
YNSTYRVVSVLTVLHQDWLNGKEYKCKVSNKGLPSS
IEKTISKAKGQPREPQVYTLPPSREEMTKNQVSLTC
LVKGFYPSDIAVEWESNGQPENNYKTTPPVLDSDGS
FFLTSKLTVDKSRWQQGNVFSCSVMHEALHNHYTQK
SLSLSPGK

The half-life extending domain may comprise an albumin-binding domain. As is known in the art, albumin has a long circulatory half-life of 19 days, due in part to its size, being above the renal threshold, and by its specific interaction and recycling via FcRn. Attachment to albumin is a well-known strategy to improve the circulatory half-life of a therapeutic molecule in vivo. Albumin may be attached non-covalently, through the use of a specific albumin binding domain, or covalently, by conjugation or direct genetic fusion. Examples of therapeutic molecules that have exploited attachment to albumin for improved half-life are given in Sleep et al., Biochim Biophys Acta. 2013 December; 1830(12):5526-34.

The albumin-binding domain may be any moiety capable of binding to albumin, including any known albumin-binding moiety. Albumin binding domains may be selected from endogenous or exogenous ligands, small organic molecules, fatty acids, peptides and proteins that specifically bind albumin. Examples of preferred albumin binding domains include short peptides, such as described in Dennis et al., J Biol Chem. 2002 Sep. 20; 277(38):35035-43 (for example the peptide QRLMEDICLPRWGCLWEDDF); proteins engineered to bind albumin such as antibodies, antibody fragments and antibody like scaffolds, for example Albudab® (O'Connor-Semmes et al., Clin Pharmacol Ther. 2014 December; 96(6):704-12), commercially provided by GSK and Nanobody® (Van Roy et al., Arthritis Res Ther. 2015 May 20; 17:135), commercially provided by Ablynx; and proteins based on albumin binding domains found in nature such as Streptococcal protein G Protein (Stork et al., Eng Des Sel. 2007 November; 20(11):569-76), for example Albumod® commercially provided by Affibody Preferably, albumin is human serum albumin (HSA). The affinity of the albumin binding domain for human albumin may be in the range of picomolar to micromolar. Given the extremely high concentration of albumin in human serum (35-50 mg/ml, approximately 0.6 mM), it is calculated that substantially all of the albumin binding domains will be bound to albumin in vivo.

The albumin-binding moiety may be linked to the C or N terminus of the other domains (i.e., the TCR or immune effector). The albumin-binding moiety may be linked to the other domains (i.e., the TCR or immune effector) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The liker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules of the invention include, but are not limited to: GGGSGGGG, GGGGS, GGGSG, GGSGG, GSGGG, GSGGGP, GGEPS, GGEGGGP, and GGEGGGSEGGGS (as described in WO2010/133828). Where the albumin-binding moiety is linked to the TCR, it may be linked to either the alpha or beta chains or both the alpha and beta chains, with or without a linker.

The multi-domain binding molecule according to the first aspect may be for use as a medicament.

In a further aspect, there is provided a pharmaceutical composition comprising the multi-domain binding molecule according to the first aspect.

In yet a further aspect, there is provided a nucleic acid encoding the multi-domain binding molecule according to the first aspect. There is also provided an expression vector comprising the nucleic acid of this aspect. In addition, there is provided a host cell comprising the nucleic acid or the vector of this aspect, wherein the nucleic acid encoding the multi-domain binding molecule is present as a single open reading frame or two distinct open reading frames encoding the alpha chain and beta china respectively.

Also provided, in a further aspect, is a method of making the multi-domain binding molecule according to the first aspect comprising maintaining the host cell described above under optional conditions for expression of the nucleic acid and isolating the multi-domain antigen binding polypeptide.

In a still further aspect there is provided a method of treatment comprising administering the multi-domain binding molecule according to the first aspect to a patient in need thereof.

Within the scope of the invention are phenotypically silent variants of any molecule disclosed herein. As used herein the term “phenotypically silent variants” is understood to refer to a variant which incorporates one or more further amino acid changes, including substitutions, insertions and deletions, in addition to those set out above, which variant has a similar phenotype to the corresponding molecule without said change(s). For the purposes of this application, phenotype comprises binding affinity (K_{D} and/or binding half-life) and specificity. Preferably, the phenotype for a soluble multi-domain binding molecule includes potency of immune activation and purification yield, in addition to binding affinity and specificity.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a molecule comprising any of the amino acid sequence described above but with one or more conservative substitutions and or one or more tolerated substitutions in the sequence, such that the amino acid sequence of the TCR has at least 90% identity, such as 90%, 91%, 92%, 93%, 94%, 95%, 96%, 97%, 98%, 99% or 100% identity, to the TCR sequences disclosed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size=3, Expect Threshold=10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the molecule, for example the TCR portion. The sequences provided at the C-terminus and/or N-terminus thereof may be truncated or extended by 1, 2, 3, 4 or 5 residues. All such variants are encompassed by the present invention.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6. The TCR sequences provided by the invention may be obtained from solid state synthesis, or any other appropriate method known in the art.

Molecules of the invention may have an ideal safety profile for use as therapeutic reagents. An ideal safety profile means that in addition to demonstrating good specificity, the molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Molecules of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or more preferably greater than 5%, or higher yield. High yield means greater than 1 mg/ml, or more preferably greater than 3 mg/ml, or greater than 5 mg/ml, or higher yield.

Methods to determine binding affinity (inversely proportional to the equilibrium constant K_{D}) and binding half life (expressed as T %) are known to those skilled in the art. In a preferred embodiment, binding affinity and binding half-life are determined using Surface Plasmon Resonance (SPR) or Bio-Layer Interferometry (BLI), for example using a BIAcore instrument or Octet instrument, respectively. It will be appreciated that doubling the affinity results in halving the K_{D}. T_{1/2} is calculated as In2 divided by the off-rate (k_{off}). Therefore, doubling of T % results in a halving in k_{off}. K_{D} and k_{off} values for TCRs are usually measured for soluble forms of the TCR, i.e. those forms which are truncated to remove cytoplasmic and transmembrane domain residues. To account for variation between independent measurements, and particularly for interactions with dissociation times in excess of 20 hours, the binding affinity and or binding half-life of a given TCR may be measured several times, for example 3 or more times, using the same assay protocol, and an average of the results taken. To compare binding data between two samples (i.e. two different TCRs and or two preparations of the same TCR) it is preferable that measurements are made using the same assay conditions (e.g. temperature). Measurement methods described in relation to TCRs may also be applied to the multi-domain antigen-binding polypeptides described herein.

Certain preferred multi-domain binding molecules of the invention are able to generate a highly potent T cell response in vitro against antigen positive cells, in particular those cells presenting low levels of antigen typical of cancer cells (i.e. in the order of 5-100, for example 50, antigens per cell (Bossi et al., (2013) Oncoimmunol. 1; 2 (11):e26840; Purbhoo et al., (2006). J Immunol 176(12): 7308-7316.)). Such TCRs may be suitable for incorporation into the multi-domain antigen-binding polypeptides described herein. The T cell response that is measured may be the release of T cell activation markers such as Interferon γ or Granzyme B, or target cell killing, or other measure of T cell activation, such as T cell proliferation. Preferably a highly potent response is one with EC₅₀ value in the nM-pM range, preferably 500 nM or lower most preferably, 1 nM of lower, or 500 pM or lower.

TCR portions of the molecules of the invention may be as heterodimers. Alpha-beta heterodimeric TCR portions of the molecules of the invention usually comprise an alpha chain TRAC constant domain sequence and/or a beta chain TRBC1 or TRBC2 constant domain sequence. The constant domains may be in soluble format (i.e. having no transmembrane or cytoplasmic domains). One or both of the constant domains may contain mutations, substitutions or deletions relative to the native TRAC and/or TRBC1/2 sequences. The term TRAC and TRBC1/2 also encompasses natural polymorphic variants, for example N to K at position 4 of TRAC (Bragado et al International immunology. 1994 February; 6(2):223-30).

The alpha and beta chain constant domain sequences may be modified by truncation or substitution to delete the native disulphide bond between Cys4 of exon 2 of TRAC and Cys2 of exon 2 of TRBC1 or TRBC2. The alpha and/or beta chain constant domain sequence(s) may have an introduced disulphide bond between residues of the respective constant domains, as described, for example, in WO 03/020763 and WO06000830. The alpha and beta constant domains may be modified by substitution of cysteine residues at position Thr 48 of TRAC and position Ser 57 of TRBC1 or TRBC2, the said cysteines forming a disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. One or both of the extracellular constant domains present in an as heterodimer may be truncated at the C terminus or C termini, for example by up to 15, or up to 10, or up to 8 or fewer amino acids. The C terminus of the alpha chain extracellular constant domain may be truncated by 8 amino acids.

TCR portions of the molecules of the invention may be in single chain format. Single chain formats include, but are not limited to, αβ TCR polypeptides of the Vα-L-Vβ, Vβ-L-Vα, Vα-Cα-L-Vβ, Vα-L-Vβ-Cβ, or Vα-Cα-L-Vβ-CP types, wherein Vα and Vβ are TCR α and β variable regions respectively, Cα and Cβ are TCR α and β constant regions respectively, and L is a linker sequence (Weidanz et al., (1998) J Immunol Methods. December 1; 221(1-2):59-76; Epel et al., (2002), Cancer Immunol Immunother. November; 51(10):565-73; WO 2004/033685; WO9918129). Single chain TCRs may have an introduced disulphide bond between residues of the respective constant domains, as described in WO 2004/033685. Single chain TCRs are further described in WO2004/033685; WO98/39482; WO01/62908; Weidanz et al. (1998) J Immunol Methods 221(1-2): 59-76; Hoo et al. (1992) Proc Natl Acad Sci USA 89(10): 4759-4763; Schodin (1996) Mol Immunol 33(9): 819-829).

Therapeutic agents which may be associated with the molecules of the invention include immune-modulators and effectors, radioactive compounds, enzymes (perforin for example) or chemotherapeutic agents (cis-platin for example). To ensure that toxic effects are exercised in the desired location the toxin could be inside a liposome linked to the multi-domain antigen-binding polypeptide described herein so that the compound is released slowly. This will prevent damaging effects during the transport in the body and ensure that the toxin has maximum effect after binding of the multi-domain antigen-binding polypeptide described herein to the relevant antigen presenting cells.

Examples of suitable therapeutic agents include, but are not limited to:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate 12arbour12ate, auristatin E vincristine and doxorubicin;
peptide cytotoxins, i.e. proteins or fragments thereof with the ability to kill mammalian cells. For example, ricin, diphtheria toxin, pseudomonas bacterial exotoxin A, Dnase and Rnase;
radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
Immuno-stimulants, i.e. immune effector molecules which stimulate immune response. For example, cytokines such as IL-2 and IFN-γ,
Superantigens and mutants thereof;
TCR-HLA fusions, e.g. fusion to a peptide-HLA complex, wherein said peptide is derived from a common human pathogen, such as Epstein Barr Virus (EBV);
chemokines such as IL-8, platelet factor 4, melanoma growth stimulatory protein, etc;
antibodies or fragments thereof, including anti-T cell or NK cell determinant antibodies (e.g. anti-CD3, anti-CD28 or anti-CD16);
antibodies or fragments thereof that bind to molecules that locate to the immune synapse
alternative protein scaffolds with antibody like binding characteristics
complement activators;
xenogeneic protein domains, allogeneic protein domains, viral/bacterial protein domains, viral/bacterial peptides.

A particularly preferred immune effector is an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, Fab fragments, F(ab′)₂ fragments, dsFv and scFv fragments, Nanobodies™ (these constructs, marketed by Ablynx (Belgium), comprise synthetic single immunoglobulin variable heavy domain derived from a camelid (e.g. camel or llama) antibody) and Domain Antibodies (Domantis (Belgium), comprising an affinity matured single immunoglobulin variable heavy domain or immunoglobulin variable light domain) or alternative protein scaffolds that exhibit antibody like binding characteristics such as Affibodies (Affibody (Sweden), comprising engineered protein A scaffold) or Anticalins (Pieris (Germany)), comprising engineered anticalins) to name but a few the immune effector is linked to the TCR portion of the multi-domain antigen binding polypeptide, where preferably the immune effector is an anti-CD3 antibody.

Linkage of the individual components of the multi-domain binding molecule may be via covalent or non-covalent attachment. Covalent attachment may be direct, or indirect via a linker sequence. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. Examples of suitable linkers that may be used in the molecules of the invention include, but are not limited to: GGGSGGGG, GGGGS, GGGSG, GGSGG, GSGGG, GSGGGP, GGEPS, GGEGGGP, and GGEGGGSEGGGS (as described in WO2010/133828).

In a further aspect, the present invention provides nucleic acid encoding a multi-domain binding molecule of the invention. In some embodiments, the nucleic acid is cDNA. In some embodiments the nucleic acid may be mRNA. The nucleic acid may be non-naturally occurring and/or purified and/or engineered. The nucleic acid sequence may be codon optimised, in accordance with expression system utilised. As is known to those skilled in the art, expression systems may include bacterial cells such as E. coli, or yeast cells, or mammalian cells, or insect cells, or they may be cell free expression systems.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a specific binding molecule of the invention forms an aspect of the present invention, as does a method of production of the specific binding molecule which method comprises expression from encoding nucleic acid therefor. Expression may conveniently be achieved by culturing under appropriate conditions recombinant host cells containing the nucleic acid. Following production by expression, a specific binding molecule may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The expression of antibodies and antibody fragments in prokaryotic cells such as E. coli is well established in the art. For a review, see for example Pluckthun, Bio/Technology 9:545-551 (1991). Expression in eukaryotic cells in culture is also available to those skilled in the art as an option for production of a specific binding molecule, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

The nucleic acid of the invention may be integrated into the genome (e.g. chromosome) of the host cell. Integration may be promoted by inclusion of sequences which promote recombination with the genome, in accordance with standard techniques.

As is well-known in the art, molecules may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34.). For the TCR portions of the molecules of the invention glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci. August; 94(8):1626-35).

For administration to patients, the molecules of the invention (preferably associated with a detectable label or therapeutic agent), nucleic acids, expression vectors or cells of the invention may be provided as part of a sterile pharmaceutical composition together with one or more pharmaceutically acceptable carriers or excipients. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

Dosages of the molecules of the present invention can vary between wide limits, depending upon the disease or disorder to be treated, the age and condition of the individual to be treated, etc. a suitable dose range for a molecule of the invention may be in the range of 25 ng/kg to 50 μg/kg or 1 μg to 1 g. A physician will ultimately determine appropriate dosages to be used.

Multi-domain antigen binding polypeptides, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

Also provided by the invention are:
A multi-domain antigen binding polypeptide, nucleic acid, pharmaceutical composition or cell of the invention for use in medicine, preferably for use in a method of treating cancer or a tumour;
the use of a multi-domain antigen binding polypeptide, nucleic acid, pharmaceutical composition or cell of the invention in the manufacture of a medicament for treating cancer or a tumour;
a method of treating cancer or a tumour in a patient, comprising administering to the patient a multi-domain antigen binding polypeptide, nucleic acid, pharmaceutical composition or cell of the invention;
an injectable formulation for administering to a human subject comprising a multi-domain antigen binding polypeptide, nucleic acid, pharmaceutical composition or cell of the invention.

The method of treatment may further include administering separately, in combination, or sequentially, an additional anti-neoplastic agent. Example of such agents are known in the art and may include immune activating agents and/or T cell modulating agents.

Preferred features of each aspect of the invention are as for each of the other aspects mutatis mutandis. The prior art documents mentioned herein are incorporated by reference to the fullest extent permitted by law.

### DESCRIPTION OF THE DRAWINGS

FIG. 1 A) Design of TCR-antiCD3-Fc fusion protein; B) Example sequence of a TCR-antiCD3-Fc fusion protein of the invention FIG. 2 shows that a TCR-antiCD3 fusion incorporating an Fc domain is able to mediate T cell activation in the presence of antigen positive target cells. Data for three IgG1-Fc fusions are shown.

FIG. 3 A) shows that a TCR-antiCD3 fusion incorporating an albumin binding peptide is able to mediate T cell activation in the presence of antigen positive target cells. Three formats are shown where in the albumin binding peptide is attached to either C-alpha (F1), N-alpha (F2) or C-beta (F3). B) shows that a TCR-antiCD3 fusion incorporating an albumin binding nanobody is able to mediate T cell activation in the presence of antigen positive target cells. Two formats are shown wherein the albumin binding peptide is attached to either C-alpha (R) or C-beta (Y). C) shows that a TCR-antiCD3 fusion incorporating an albumin binding domain antibody (Albudab®) is able to mediate T cell activation in the presence of antigen positive target cells. One format is shown wherein the Albudab® is attached to C-alpha of the TCR-antiCD3 fusion.

FIG. 4 A) Shows PK characteristics of TCR-antiCD3-Albudab® fusion in mouse serum and B) shows theoretical PK in humans for a TCR-antiCD3-Albudab® fusion based assessment in mouse.

### EXAMPLES

The following examples describe multi-domain binding molecules of the invention, which may be referred to as TCR-antiCD3-Fc fusion proteins or TCR-antiCD3-albumin-binding fusion proteins.

### Example 1 (Fc Fusion)

a) Design of TCR-antiCD3-Fc Fusion Proteins

In this example, a TCR-antiCD3 fusion protein comprising a high affinity TCR that binds to a HLA-A*02 restricted peptide from PRAME was used. Examples of such molecules are provided in WO2018234319.

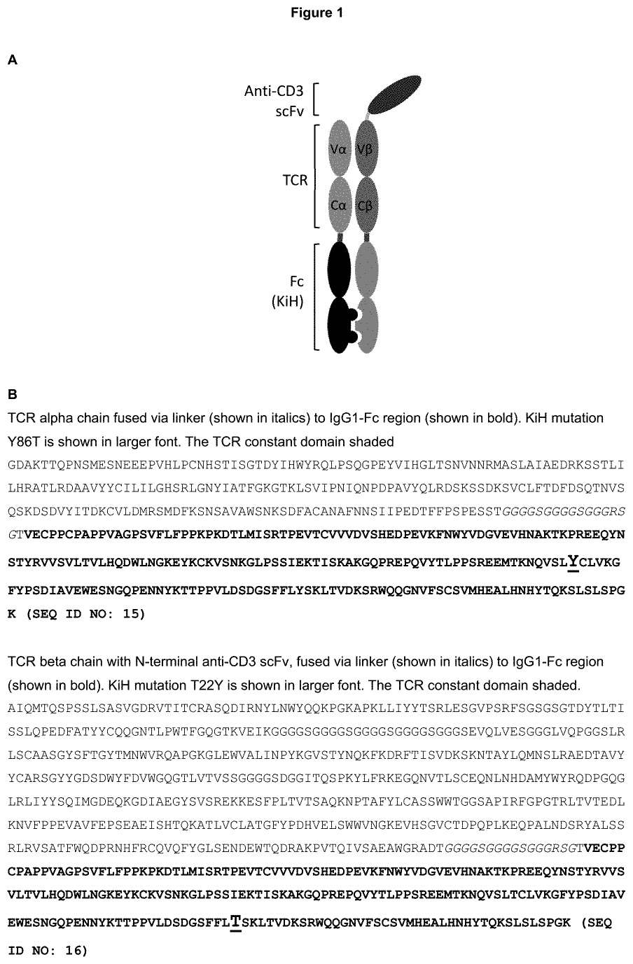

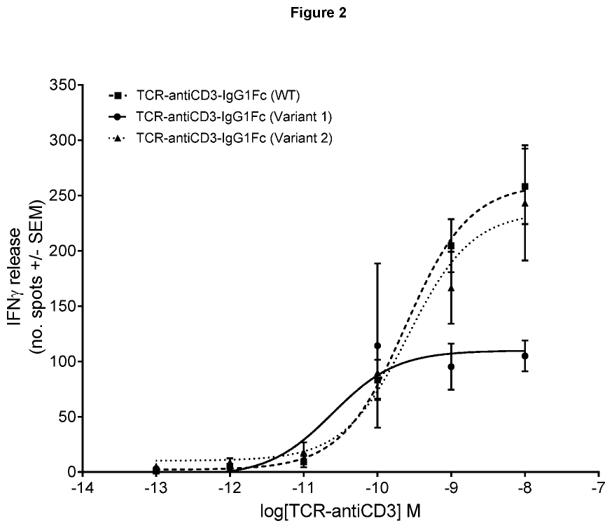

The human IgG1 Fc domain was fused via a linker to the C terminus of the TCR-antiCD3 (see FIG. 1A for schematic). A further two constructs were made comprising functional variants of human IgG1 Fc that are known in the art. Variant 1 does not bind to Fcγ receptors (FcγRs) or complement protein C1q and is therefore functionally silent. Variant 2 shows increased binding to FcRn, which may result in extended in vivo half-life. In each case the Fc domain comprises known knobs in holes mutations Y86T and T22Y to facilitate heterodimerization.

FIG. 1B shows the sequence of a TCR-anti-CD3-Fc fusion comprising a functionally silent human IgG1 Fc (Variant 1)

b) Expression and Purification of TCR-antiCD3-Fc Fusion Proteins

Expression of Fc fusions was performed using a transient expression system based on suspension-adapted Chinese Hamster Ovary (CHO) cells (ExpiCHO Expression system, Thermo Fisher). Cells were transfected according to the manufacturer's instructions, using mammalian expression plasmids containing the relevant TCR chains fused to various Ig Fc domains. Following the harvest, cell culture supernatants were clarified by centrifuging of the cells at 4000-5000×g for 30 minutes in a refrigerated centrifuge. Supernatants were filtered through a 0.22-μm filter and collected for further purification.

For purification, Fc fusions were first adjusted with buffer before being purified using mAbselect Sure prepacked columns (GE Healthcare or equivalent resins) as per the manufacturer's guidelines. Specific protein containing fractions were pooled and further purified by size exclusion chromatography using appropriate columns (GE Healthcare) in physiologically-relevant buffers. Specific protein containing fractions were pooled and concentrated for downstream testing and storage.

c) Potent T Cell Activation by TCR-antiCD3-Fc Fusion Proteins

The TCR-antiCD3-Fc fusion proteins were assessed for their ability to mediate potent redirection of CD3+ T cells against antigen presenting T2 cells. Interferon-γ (IFN-γ) release was used as a read out for T cell activation.

Assays were performed using a human IFN-γ ELISPOT kit (BD Biosciences) according to the manufacturer's instructions. Briefly, T2 cells were used as target cells and pulsed with 5 nM PRAME peptide. Target cells were prepared at a density of 1×10⁶/ml in assay medium (RPMI 1640 containing 10% heat inactivated FBS and 1% penicillin-streptomycin-L-glutamine) and plated at 50,000 cells per well in a volume of 50 μl. Peripheral blood mononuclear cells (PBMC), isolated from fresh donor blood, were used as effector cells and plated at 35,000 cells per well in a volume of 50 μl. TCR-antiCD3-Fc fusion proteins were titrated to final concentrations of between 10 nM and 0.0001 nM, and added to the well in a volume of 50 μl.

Plates were prepared according to the manufacturer's instructions. Wells containing target cells, effector cells and fusion proteins were made up to a final volume of 200 μl with assay medium. All reactions were performed in triplicate. Control wells were also prepared with the omission of, either fusion protein, effector cells, or target cells. The plates were incubated overnight (37° C./5% CO₂). The next day the plates were washed three times with wash buffer (1×PBS sachet, containing 0.05% Tween-20, made up in deionised water). Primary detection antibody was then added to each well in a volume of 50 μl. Plates were incubated at room temperature for 2 hours prior to being washed again three times. Secondary detection was performed by adding 50 μl of diluted streptavidin-HRP to each well and incubating at room temperature for 1 hour and the washing step repeated. No more than 15 mins prior to use, one drop (20 μl) of AEC chromogen was added to each 1 ml of AEC substrate and mixed and 50 μl added to each well. Spot development was monitored regularly and plates were washed in tap water to terminate the development reaction. The plates were then allowed to dry at room temperature for at least 2 hours prior to counting the spots using a CTL analyser with Immunospot software (Cellular Technology Limited). Data were prepared and analysed using PRISM software.

Results presented in FIG. 2 demonstrate that TCR-antiCD3-Fc fusion proteins mediate effective T cell activation in the presence of antigen positive target cells. Ec50 values were in the pM range (238 pM 257 pM and 25 pM respectively for fusions to IgG1-Fc, IgG1-Fc-variant1 and IgG1-Fc-variant2 respectively). Control experiments with antigen negative target cells demonstrated that the functionally silent IgG1-variant2 gave negligible background activity. Functionally silent Fc domains were therefore considered most preferable for therapeutic use.

### Example 2 (Albumin Binding)

a) Design of TCR-antiCD3-Albumin-Binding Fusion Proteins

In a first design, the TCR-antiCD3 fusion protein comprised a high affinity TCR that binds to a HLA-A*02 restricted peptide from gp100. The amino acid sequence of such molecules is disclosed in WO2011001152. Specifically, the TCR-antiCD3 fusion comprised the alpha chain of SEQ ID No: 45 of WO2011001152, wherein amino acids 1-109 are replaced with SEQ ID No. 8 of WO2011001152, and the amino acid at position 1 is A, based on the numbering of SEQ ID No: 45; and a beta chain of SEQ ID No: 36 of WO2011001152, in which residues 259-370 correspond to SEQ ID No. 27 of WO2011001152, amino acids at position 1 and 2 are A and I respectively. An albumin binding peptide having the amino acid sequence QRLMEDICLPRWGCLWEDDF (as described in Dennis et al., J Biol Chem. 2002 Sep. 20; 277(38):35035-43) was attached to the TCR-antiCD3 fusion via a linker. A suitable linker is GGGGS. Three variants were prepared in which the albumin binding peptide was fused at three different attachment sites: C-alpha (F1), N-alpha (F2) or C-beta (F3).

In a second design, an albumin binding nanobody was attached to the same TCR-antiCD3 fusion as used in the first design. An albumin binding nanobody having the sequence of SEQ ID No: 52 in WO2006122787 was attached to TCR-antiCD3 fusion via a linker. A suitable linker is GGGGS. Two variants were prepared in which the albumin binding nanobody was fused at two different attachment sites: C-alpha (R) or C-beta (Y).

In a third design, an albumin binding domain antibody was attached to the C terminus of the TCR-antiCD3 fusion alpha chain. The antibody belongs to the Albudab® platform. Two variants of the domain antibody were used; DOM 7h-10-14 dAb and DOM 7h-11-15 dAb, provided by SEQ ID Nos: 26 and 27 respectively in WO201010893. The antibody was attached via a linker and directly (i.e. without a linker). A suitable linker is GGGGS. The TCR-antiCD3 fusion protein comprised a high affinity TCR that binds to a HLA-A*01 restricted peptide from MAGEA3. Amino acid sequences of such molecules are provided in WO2013041865.

b) Expression and Purification of TCR-antiCD3-Albumin-Binding Fusion Proteins

TCR-antiCD3-albumin-binding fusion proteins were expressed in E. coli as inclusion bodies and subsequently refolded and purified, using the same methodology as known in the art for TCR-antiCD3 fusion proteins (for example, see WO2011001152, example 2)

c) Potent T Cell Activation by TCR-antiCD3-Albumin-Binding Fusion Proteins

TCR-antiCD3-albumin binding fusion proteins were assessed for their ability to mediate potent redirection of CD3+ T cells against antigen positive cancer cells. Interferon-γ (IFN-γ) release was used as a read out for T cell activation.

Assays were performed using a human IFN-γ ELISPOT kit (BD Biosciences) according to the manufacturer's instructions. Briefly, for fusions comprising albumin binding peptide, melanoma Mel526 cells were used as target cells. Target cells were prepared at a density of 1×10⁶/ml in assay medium (RPMI 1640, plus 150 μM human serum albumin (HSA) and 1% penicillin-streptomycin-L-glutamine) and plated at 50,000 cells per well in a volume of 50 μl. Peripheral blood mononuclear cells (PBMC), isolated from fresh donor blood, were used as effector cells and plated at 30,000 cells per well in a volume of 50 μl. TCR-antiCD3-Fc fusion proteins were titrated to final concentrations of between 10 nM, and 0.0001 nM, and added to the well in a volume of 50 μl.

For fusions comprising Albudab®, myeloma EJM cells were used as target cells. Target cells were prepared at a density of 1×10⁶/ml in assay medium (RPMI 1640, plus 45 μM HSA, and 1% penicillin-streptomycin-L-glutamine) and plated at 50,000 cells per well in a volume of 50 μl. Peripheral blood mononuclear cells (PBMC), isolated from fresh donor blood, were used as effector cells and plated at 30,000 cells per well in a volume of 50 μl. TCR-antiCD3-Fc fusion proteins were titrated to final concentrations of between 10 nM, and 0.0001 nM, and added to the well in a volume of 50 μl.

Plates were prepared and developed as described in Example 1c

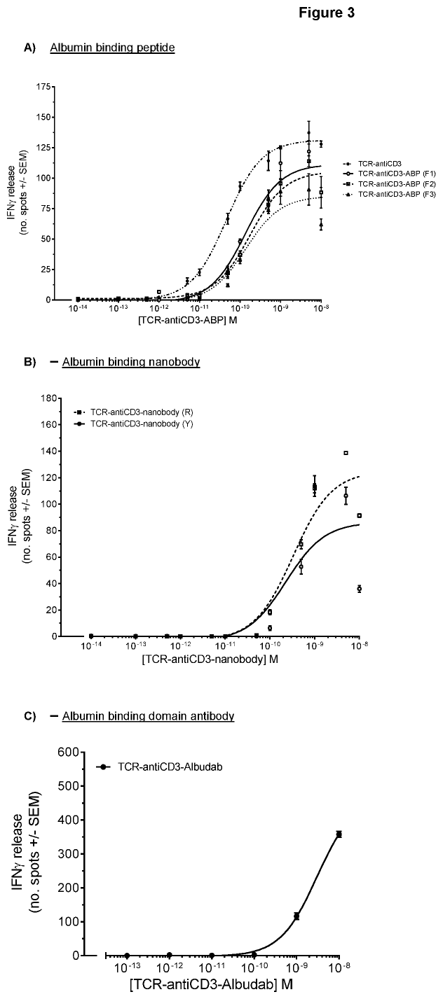

FIG. 3A-C shows that fusion of an albumin binding moiety to a TCR-antiCD3 fusion protein, mediates effective T cell activation in the presence of antigen positive target cells. Ec50 values were in the pM range (137.4/178.0/137.1)

### Example 3 (Extended Half-Life)

a) PK Assessment of TCR-antiCD3-Albumin-Binding Fusion Proteins

The PK characteristics of TCR-antiCD3-AlbudAb fusions were investigated in mouse serum.

Mice were dose with 0.1 mg/kg of fusion protein by intravenous bolus injection and serum samples were taken at regular intervals over a period of 120 hours. PK assessment was carried out by using an ELISA based assay. In brief, biotinylated pHLA complex was attached to streptavidin-coated plates and serum samples were then added. A detection step was carried out using a primary goat antibody against anti-CD3 scFv and a HRP-conjugated anti-goat IgG activated for colourimetric detection with TMB at 450 nm. Results generated were used to confirm the presence and binding activity of the TCR-antiCD3-Albudab fusion by using a dilution series and analysis against a standard curve. The results are reported as % activity and used to generate a plot of Cmax over time.

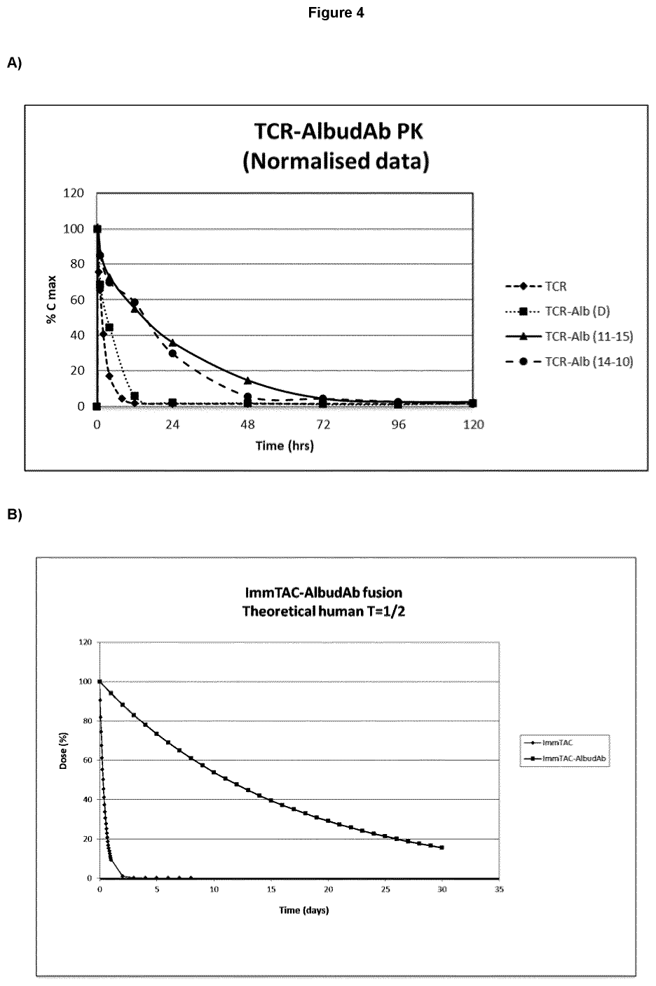

The resulting PK data are shown in FIG. 4A. Note that the samples denoted ‘TCR-Alb (10-14)’ and ‘TCR-Alb (11-15)’ refer to the TCR-antiCD3 fusions described in Example 2a. ‘TCR-Alb (D)’ is a control sample fused to a non-albumin binding Albudab, and ‘TCR’ refers to the TCR-antiCD3 without Albudab.

The PK data from FIG. 4A were used to calculate a theoretical PK profile for TCR-antiCD3-AlbudAb fusions in humans (FIG. 4B). Fusion to Albudab is predicted to extend in-vivo half life from 7 h to 264 h.

1. A multi-domain binding molecule comprising:
i) a peptide-major histocompatibility complex (pMHC) binding moiety linked to a T cell engaging immune effector; and
ii) a half-life extending domain, comprising an immunoglobulin Fc or an albumin binding domain.

2. A multi-domain binding molecule according to claim 1, wherein the pMHC binding moiety is a T cell receptor (TCR) or TCR-like antibody, comprising TCR and/or antibody variable domains, and at least one constant domain.

3. A multi-domain binding molecule according to claim 2, wherein the TCR is a heterodimeric alpha/beta TCR polypeptide pair.

4. A multi-domain binding molecule according to claim 2, wherein the TCR is a single chain alpha/beta TCR polypeptide.

5. A multi-domain binding molecule according to claim 4, wherein the T-cell engaging immune effector domain is a CD3 effector domain that activates a T cell through interaction with CD3 and or TCR/CD3 complex.

6. A multi-domain binding molecule according to claim 5, wherein the CD3 effector domain comprises an antibody scFv or antibody-like scaffold.

7. A multi-domain binding molecule according to claim 6, wherein the half-life extending domain is an immunoglobulin Fc domain.

8. A multi-domain binding molecule according to claim 6, wherein the half-life extending domain comprises an albumin binding domain.

9. A multi-domain binding molecule according to claim 1, wherein the half-life extending domain is linked to the C or N terminus of the pMHC binding moiety or to the C or N terminus of the T cell engaging immune effector.

10. A multi-domain binding molecule according to claim 1, wherein the half-life extending domain is linked to the pMHC binding moiety or to the T cell engaging immune effector via a linker.

11. A multi-domain binding molecule according to claim 1, for use as a medicament.

12. A pharmaceutical composition comprising the multi-domain binding molecule according to claim 1.

13. Nucleic acid encoding the multi-domain binding molecule according to claim 1.

14. An expression vector comprising nucleic acid of claim 13.

15. A host cell comprising nucleic acid of claim 13, wherein the nucleic acid encoding the multi-domain binding molecule is present as a single open reading frame or two distinct open reading frames encoding the alpha chain and beta chain respectively.

16. A method of making the multi-domain binding molecule according to claim 1 comprising maintaining the host cell of claim 15 under optional conditions for expression of the nucleic acid and isolating the multi-domain antigen binding polypeptide.

17. A method of treatment comprising administering the multi-domain binding molecule of claim 1 to a patient in need thereof.
